SEQUENCE LISTING (1 sequence)

SEQ ID NO: 1 - RNA, 19 nt, Artificial Sequence
  siGL2-5′
cguacgcggaauacuucga

(19) United States
(12) Patent Application Publication
(10) Pub. No.: US 2022/0265606 A1
(43) Pub. Date: Aug. 25, 2022

(54) ANTICANCER AGENTS

(71) Applicant: The Texas A&M University System, College Station, TX (US)

(72) Inventors: Stephen Safe, College Station, TX (US); Xi Li, Richardson, TX (US); Keshav Karki, College Station, TX (US)

(21) Appl. No.: 17/631,390

(22) PCT Filed: Jul. 31, 2020
(86) PCT No.: PCT/US2020/044630

§ 371 Date: Jan. 28, 2022

Related U.S. Application Data
(60) Provisional application No. 62/880,801, filed on Jul. 31, 2019.

(51) Int. Cl. A61K 31/404

(52) U.S. Cl. CPC A61K 31/404

(57) ABSTRACT

In an embodiment, the present disclosure pertains to a method of treating a disease by induction of activity in cells. Generally, the method includes administering a bis-indole-derived compound to a subject in need thereof. In some embodiments, the method further includes binding, by the bis-indole-derived compound, to at least one of nuclear receptor 4A1 (NR4A1) and nuclear receptor 4A2 (NR4A2). In another embodiment, the present disclosure pertains to a compound for treating a disease by induction of activity in cells. Generally, the compound includes a bis-indole-derived compound. In some embodiments, the bis-indole-derived compound binds to at least one of NR4A1 and NR4A2.

### CROSS-REFERENCE TO RELATED APPLICATIONS

This patent application claims priority from, and incorporates by reference the entire disclosure of, U.S. Provisional Application No. 62/880,801 filed on Jul. 31, 2019.

### TECHNICAL FIELD

The present disclosure relates generally to anticancer agents and more particularly, but not by way of limitation, to NR4A2 ligands as anticancer agents.

### BACKGROUND

This section provides background information to facilitate a better understanding of the various aspects of the disclosure. It should be understood that the statements in this section of this document are to be read in this light, and not as admissions of prior art.

Nuclear receptor 4A2 (NR4A2) is an orphan nuclear receptor that is expressed in many cell types and is induced by stress and overexpressed in tumors. Initially, the bis-indole-derived compounds 1,1-bis(3′indolyl)-1-(p-chlorophenyl)methane (DIM-C-pPhCl) and its para-bromo analog (DIM-C-pPhBr) were identified as NR4A2 antagonists and demonstrated that these compounds induced vasoactive intestinal peptide (VIP) in pancreatic cancer cells. Using this assay, the present disclosure identifies other bis-indole-derived compounds as inducers of VIP in pancreatic cancer cells and identifies a “second generation” set of NR4A2 ligands. Moreover, it has been identified that certain patient-derived glioblastoma cells express NR4A2 and that NR4A2 is pro-oncogenic and inhibited by bis-indole-derived NR4A2 ligands.

There are currently no drugs available for targeting NR4A2 in cancer or other NR4A2-dependent anti-inflammatory diseases and the bis-indole-derived compounds disclosed herein would be unique and well-suited for targeting NR4A2 in cancer. Thus, the present disclosure describes bis-indole-derived ligands and their role as NR4A2 antagonists that exhibit a broad spectrum of anticancer activities. The bis-indole-derived NR4A2 ligands disclosed herein can be used for development of anticancer drugs targeting NR4A2 that are highly advantageous for glioblastoma patients who currently have a dismal prognosis for survival.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method of treating a disease by induction of activity in cells. Generally, the method includes administering a bis-indole-derived compound to a subject in need thereof. In some embodiments, the method further includes binding, by the bis-indole-derived compound, to at least one of nuclear receptor 4A1 (NR4A1) and nuclear receptor 4A2 (NR4A2). In some embodiments, the induction of activity in the cells is at least one of anticancer activity and anti-inflammatory activity. In some embodiments, the bis-indole-derived compound (CDIM) includes two or more substituents on a phenyl ring thereof. In some embodiments, the bis-indole-derived compound includes, without limitation, 1,1-bis(3′-indolyl)-1-(p-chlorophenyl)methane (DIM-C-pPhCl; 4-Cl), 1,1-bis(3′-indolyl)-1-(4-chloro-3-trifluoromethylphenyl)methane (3-CF₃-4-Cl), 1,1-dimethyl-1,1-bis(3′-indolyl)-1-(p-hydroxyphenyemethane (N—Me—4-OH), 1,1-bis(3′-indolyl)-1-(4-bromo-2-hydroxy-phenyl)methane (2-OH-4-Br), 1-bis(3′indolyl)-1-(p-bromophenyl)methane (DIM-C-pPhBr), 1,1-bis(3′-indolyl)-1-(p-hydroxyphenyl)methane (CDIM8), a 3,5-disubstituted analog of CDIM8, CDIM8-3,5-(CH₃)₂, CDIM8-3,5-Bra, CDIM8-3,5-Cl₂, CDIM8-3-Br-5-OCH₃, CDIM8-3-Cl-5-OCH₃, CDIM8-3-Cl-5-Br, CDIM8-3-Cl-5-F, CDIM, a 3,5-disubstituted analog of CDIM, CDIM-3,5-Br₂, CDIM-2,5-Br₂, CDIM-3,5-Cl₂, CDIM-3,5-(CH₃)₂, CDIM-3-Br-5-OCH₃, CDIM-3-Br-5-OCH₃, CDIM-3-Cl-5-OCF₃, CDIM-3-Cl-5-OCF₃, CDIM-3-Cl-5-CF₃, and combinations thereof.

In some embodiments, the bis-indole-derived compound performs a function on the cells including, without limitation, inducing NR4A1-dependent transactivation in the cells, inducing NR4A2-dependent transactivation in the cells, inhibiting growth of the cells, inducing apoptosis in the cells, inhibiting survival of the cells, inhibiting migration of the cells, and combinations thereof. In some embodiments, the cells include, without limitation, A172, U87-MG, U98G, CCF-STTG1, 1708, 15037, 14004s, 14015s, 15049, glioblastoma multiforme (GBM) cells, and combinations thereof. In some embodiments, the bis-indole-derived compound is at least one of a bis-indole-derived NR4A1 ligand and a bis-indole-derived NR4A2 ligand. In some embodiments, the at least one of the bis-indole-derived NR4A1 ligand and the bis-indole-derived NR4A2 ligand performs a function including, without limitation, antagonizing NR4A1 in the cancer cells, targeting NR4A1 in the cancer cells, antagonizing NR4A2 in the cancer cells, targeting NR4A2 in the cancer cells, and combinations thereof. In some embodiments, the cells include at least one of NR4A1 and NR4A2 in cancer cells. In some embodiments, the cancer cells correspond to a cancer including, without limitation, brain cancer, breast cancer, kidney cancer, colon cancer, pancreatic cancer, lung cancer, and combinations thereof. In some embodiments, the disease includes, without limitation, cancer, brain cancer, breast cancer, kidney cancer, colon cancer, pancreatic cancer, lung cancer, an inflammatory disease, asthma, chronic peptic ulcers, tuberculosis, rheumatoid arthritis, periodontitis, ulcerative colitis, Crohn's disease, sinusitis, active hepatitis, and combinations thereof.

In an additional embodiment, the present disclosure pertains to a method of inducing anticancer activity in a tumor. Generally, the method includes administering a bis-indole-derived compound to a subject in need thereof. In some embodiments, the method further includes binding, by the bis-indole-derived compound, to at least one of nuclear receptor 4A1 (NR4A1) and nuclear receptor 4A2 (NR4A2). In some embodiments, the bis-indole-derived compound (CDIM) includes two or more substituents on a phenyl ring thereof. In some embodiments, the bis-indole-derived compound includes, without limitation, 1,1-bis(3′-indolyl)-1-(p-chlorophenyl)methane (DIM-C-pPhCl; 4-Cl), 1,1-bis(3′-indolyl)-1-(4-chloro-3-trifluoromethylphenyl)methane (3 -CF₃-4-Cl), 1,1-dimethyl-1,1-bis(3′-indolyl)-1-(p-hydroxyphenyl)methane (N—Me-4-OH), 1,1-bis(3′-indolyl)-1-(4-bromo-2-hydroxy-phenyl)methane (2-OH-4-Br), 1-bis(3′indolyl)-1-(p-bromophenyl)methane (DIM-C-pPhBr), 1,1-bis(3′-indolyl)-1-(p-hydroxyphenyl)methane (CDIM8), a 3,5-disubstituted analog of CDIM8, CDIM8-3,5-(CH₃)₂, CDIM8-3,5-Bra, CDIM8-3,5-Cl₂, CDIM8-3-Br-5-OCH₃, CDIM8-3-Cl-5-OCH₃, CDIM8-3-Cl-5-Br, CDIM8-3-Cl-5-F, CDIM, a 3,5-disubstituted analog of CDIM, CDIM-3,5-Br₂, CDIM-2,5-Br₂, CDIM-3,5-Cl₂, CDIM-3,5-(CH₃)₂, CDIM-3-Br-5-OCF₃, CDIM-3-Br-5-OCH₃, CDIM-3-Cl-5-OCH₃, CDIM-3-Cl-5-OCF₃, CDIM-3-Cl-5-CF₃, and combinations thereof.

In some embodiments, the bis-indole-derived compound performs a function on cells of the tumor including, without limitation, inducing NR4A1-dependent transactivation in the cells, inducing NR4A2-dependent transactivation in cells of the tumor, inhibiting growth of cells of the tumor, inducing apoptosis in cells of the tumor, inhibiting survival of cells of the tumor, inhibiting migration of cells of the tumor, and combinations thereof. In some embodiments, the tumor includes cells including, without limitation, A172, U87-MG, U98G, CCF-STTG1, 1708, 15037, 14004s, 14015s, 15049, glioblastoma multiforme (GBM) cells, and combinations thereof. In some embodiments, the bis-indole-derived compound is at least one of a bis-indole-derived NR4A1 ligand and a bis-indole-derived NR4A2 ligand. In some embodiments, the at least one of the bis-indole-derived NR4A1 ligand and the bis-indole-derived NR4A2 ligand performs a function including, without limitation, antagonizing NR4A1 in cells of the tumor, targeting NR4A1 in cells of the tumor, antagonizing NR4A2 in cells of the tumor, targeting NR4A2 in cells of the tumor, and combinations thereof. In some embodiments, cells of the tumor are cancerous. In some embodiments, the tumor includes, without limitation, a brain tumor, a breast tumor, a kidney tumor, a colon tumor, a pancreatic tumor, a lung tumor, and combinations thereof.

In a further embodiment, the present disclosure pertains to a method of inducing anticancer activity in glioblastoma multiforme (GBM) cells. Generally, the method includes administering a bis-indole-derived compound to a subject in need thereof. In some embodiments, the method further includes binding, by the bis-indole-derived compound, to at least one of nuclear receptor 4A1 (NR4A1) and nuclear receptor 4A2 (NR4A2). In some embodiments, the bis-indole-derived compound (CDIM) includes two or more substituents on a phenyl ring thereof. In some embodiments, the bis-indole-derived compound includes, without limitation, 1,1-bis(3′-indolyl)-1-(p-chlorophenyl)methane (DIM-C-pPhCl; 4-Cl), 1,1-bis(3′-indolyl)-1-(4-chloro-3-trifluoromethylphenyl)methane (3-CF₃-4-Cl), 1,1-dimethyl-1,1-bis(3′-indolyl)-1-(p-hydroxyphenyemethane (N—Me-4-OH), 1,1-bis(3′-indolyl)-1-(4-bromo-2-hydroxy-phenyl)methane (2-OH-4-Br), 1-bis(3′indolyl)-1-(p-bromophenyl)methane (DIM-C-pPhBr), 1,1-bis(3′-indolyl)-1-(p-hydroxyphenyl)methane (CDIM8), a 3,5-disubstituted analog of CDIM8, CDIM8-3,5-(CH₃)₂, CDIM8-3,5-Bra, CDIM8-3,5-Cl₂, CDIM8-3-Br-5-OCH₃, CDIM8-3-Cl-5-OCH₃, CDIM8-3-Cl-5-Br, CDIM8-3-Cl-5-F, CDIM, a 3,5-disubstituted analog of CDIM, CDIM-3,5-Bra, CDIM-2,5-Bra, CDIM-3,5-Cl₂, CDIM-3,5-(CH₃)₂, CDIM-3-Br-5-OCF₃, CDIM-3-Br-5-OCH₃, CDIM-3-Cl-5-OCH₃, CDIM-3-Cl-5-OCF₃, CDIM-3-Cl-5-CF₃, and combinations thereof.

In some embodiments, the bis-indole-derived compound performs a function on the GBM cells including, without limitation, inducing NR4A1-dependent transactivation in the GBM cells, inducing NR4A2-dependent transactivation in the GBM cells, inhibiting growth of the GBM cells, inducing apoptosis in the GBM cells, inhibiting survival of the GBM cells, inhibiting migration of the GBM cells, and combinations thereof. In some embodiments, the GBM cells include, without limitation, A172, U87-MG, U98G, CCF-STTG1, and combinations thereof. In some embodiments, the bis-indole-derived compound is at least one of a bis-indole-derived NR4A1 ligand and a bis-indole-derived NR4A2 ligand. In some embodiments, the at least one of the bis-indole-derived NR4A1 ligand and the bis-indole-derived NR4A2 ligand performs a function including, without limitation, antagonizing NR4A1 in the GBM cells, targeting NR4A1 in the GBM cells, antagonizing NR4A2 in the GBM cells, targeting NR4A2 in the GBM cells, and combinations thereof. In some embodiments, the GBM cells are cancerous cells. In some embodiments, the cancerous cells include, without limitation, brain cancer cells, breast cancer cells, kidney cancer cells, colon cancer cells, pancreatic cancer cells, lung cancer cells, and combinations thereof.

In another embodiment, the present disclosure pertains to a compound for treating a disease by induction of activity in cells. Generally, the compound includes a bis-indole-derived compound. In some embodiments, the bis-indole-derived compound binds to at least one of nuclear receptor 4A1 (NR4A1) and nuclear receptor 4A2 (NR4A2). In some embodiment, the induction of activity in the cells is at least one of anticancer activity and anti-inflammatory activity. In some embodiments, the bis-indole-derived compound (CDIM) includes two or more substituents on a phenyl ring thereof. In some embodiments, the bis-indole-derived compound includes, without limitation, 1,1-bis(3′-indolyl)-1-(p-chlorophenyl)methane (DIM-C-pPhCl; 4-Cl), 1,1-bis(3′-indolyl)-1-(4-chloro-3-trifluoromethylphenyl)methane (3-CF₃-4-Cl), 1,1-dimethyl-1,1-bis(3′-indolyl)-1-(p-hydroxyphenyemethane (N—Me-4-OH), 1,1-bis(3′-indolyl)-1-(4-bromo-2-hydroxy-phenyl)methane (2-OH-4-Br), 1-bis(3′indolyl)-1-(p-bromophenyl)methane (DIM-C-pPhBr), 1,1-bis(3′-indolyl)-1-(p-hydroxyphenyl)methane (CDIM8), a 3,5-disubstituted analog of CDIM8, CDIM8-3,5-(CH₃)₂, CDIM8-3,5-Bra, CDIM8-3,5-Cl₂, CDIM8-3-Br-5-OCH₃, CDIM8-3-Cl-5-OCH₃, CDIM8-3-Cl-5-Br, CDIM8-3-Cl-5-F, CDIM, a 3,5-disubstituted analog of CDIM, CDIM-3,5-Br₂, CDIM-2,5-Br₂, CDIM-3,5-Cl₂, CDIM-3,5-(CH₃)₂, CDIM-3-Br-5-OCF₃, CDIM-3-Br-5-OCH₃, CDIM-3-Cl-5-OCH₃, CDIM-3-Cl-5-OCF₃, CDIM-3-Cl-5-CF₃, and combinations thereof.

In some embodiments, the bis-indole-derived compound performs a function including, without limitation, inducing NR4A1-dependent transactivation in cells, NR4A2-dependent transactivation in cells, inhibiting growth of cells, inducing apoptosis in cells, inhibiting survival of cells, inhibiting migration of cells, and combinations thereof. In some embodiments, the cells include, without limitation, A172, U87-MG, U98G, CCF-STTG1, 1708, 15037, 14004s, 14015s, 15049, glioblastoma multiforme (GBM) cells, and combinations thereof. In some embodiments, the cells include at least one of NRA1 and NR4A2 in cells. In some embodiments, the cells are cancer cells. In some embodiments, the cancer cells include, without limitation, brain cancer cells, breast cancer cells, kidney cancer cells, colon cancer cells, pancreatic cancer cells, lung cancer cells, and combinations thereof. In some embodiments, the bis-indole-derived compound is at least one of a bis-indole-derived NR4A1 ligand and a bis-indole-derived NR4A2 ligand. In some embodiments, the at least one of the bis-indole-derived NR4A1 ligand and the bis-indole-derived NR4A2 ligand performs a function including, without limitation, antagonizing NR4A1, targeting NR4A1, antagonizing NR4A2, targeting NR4A2, and combinations thereof. In some embodiments, the disease includes, without limitation, cancer, brain cancer, breast cancer, kidney cancer, colon cancer, pancreatic cancer, lung cancer, an inflammatory disease, asthma, chronic peptic ulcers, tuberculosis, rheumatoid arthritis, periodontitis, ulcerative colitis, Crohn's disease, sinusitis, active hepatitis, and combinations thereof.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIGS. 1A-1D illustrate effects of bis-indole analogs and quinoline derivatives on vasoactive intestinal peptide (VIP) gene expression;

FIGS. 2A-2C illustrate nuclear receptor 4A (NR4A) 2 expression and function in glioblastoma cells;

FIGS. 3A-3C illustrate expression and function of NR4A3;

FIGS. 4A-C illustrates NR4A2 ligand dependent effects on transactivation;

FIGS. 5A-5 D illustrate NR4A2 antagonist-induced responses;

FIGS. 6A-6C illustrate NR4A2 antagonists induce apoptosis in glioblastoma cells;

FIGS. 7A-B illustrate NR4A2 antagonists inhibit migration/invasion and glioblastoma tumor growth;

FIG. 8 illustrates binding of 2,4-dichlorophenyl analog to NR4A1 and NR4A2;

FIG. 9 illustrates binding of 3-chloro-5-methoxyphenyl analog to NR4A1 and NR4A2;

FIG. 10 illustrates a prototypical NR4A2 ligand.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

The orphan nuclear receptor 4A (NR4A1) family contains three receptors, NR4A1 (Nur77), NR4A2 (Nurr1), and NR4A3 (Nor1), which exhibit significant structural similarities in their ligand binding domains (LBDs) and DNA BDs, whereas their N-terminal (A/B) domains containing activation function 1 (AF1) are highly divergent. The initial discovery of NR4A receptors was linked to their rapid induction by multiple stimuli in various tissues/cells and organs. These responses play important roles in coping with both exogenous and endogenous stressors and the tissue-specific expression and induction of NR4A receptors that contributes to their specificity. Ongoing studies have identified multiple roles for NR4A receptors in maintaining cellular homeostasis and in pathophysiology, including, but not limited to, cancer. Initial studies in knockout mouse models showed that combined loss of NR4A1 and NR4A3 resulted in development of acute myeloid leukemia in mice, suggesting tumor suppressor-like activity for these receptors on leukemia. In contrast, there is extensive evidence that NR4A1 is highly expressed in most solid tumors and overexpression of NR4A1 in tumors from lung, colon and breast cancer patients is a negative prognostic factor, whereas less is known about the functions of NR4A2 and NR4A3 in solid tumors. Ongoing studies in breast, kidney, colon, pancreatic and lung cancer and rhabdomyosarcoma (RMS) cells show that NR4A1 plays an important role in cancer cell growth, survival and migration/invasion through regulation of genes that drive these responses. Moreover, recent studies show that transforming growth factor β (TGFβ)-induced invasion of breast and lung cancer cells is also NR4A1-dependent and is due to nuclear export of the receptor which facilitates proteasome-dependent degradation of SMAD7.

The role of NR4A2 in cancer and the effects of synthetic NR4A2 ligands are not well defined, although most existing data suggest that like NR4A1, NR4A2 is also pro-oncogenic in most cancer cell lines. Moreover, in many of these tumors, NR4A2 is a negative prognostic factor for patient survival, and the overall profile of NR4A2 and NR4A1 in the various types of cancer is similar. Both orphan receptors also bind and inactivate p53.

NR4A2 has been extensively characterized in subcellular regions in the brain, and NR4A2^{-/-} mice do not generate mid-brain dopaminergic neurons and die soon after birth. Several laboratories have been investigating the role of NR4A2 in Parkinson's disease, and studies have demonstrated that the NR4A2 agonist 1,1-bis(3′-indolyl)-1-(p-chlorophenyl)methane [DIM-C-pPhCl (C-DIM12)] crosses the blood-brain barrier and accumulates in the brain, and in vivo studies showed that DIM-C-pPhCl inhibited 1-methyl-4-phenyl-1,2,3,6-tetrahydropyridine (MPTP)-induced loss of dopaminergic neurons and other markers of neurodegeneration.

The expression of NR4A receptors and the potential role of ligands for these receptors in glioblastomas and other neuronal tumors have not been investigated, although one study showed drug-induced expression of NR4A1 in a glioblastoma multiforme (GBM) cell line. Therefore, NR4A expressions were initially screened for in several established GBM cell lines and five patient-derived GBM cell lines. Western blot analysis of cell lysates showed that four established cell lines expressed NR4A1, NR4A2 and NR4A3; in the patient-derived cells, there was variable expression of NR4A1 and NR4A3, whereas NR4A2 was highly expressed in all five cell lines. Thus, glioblastoma cells serve as an ideal model for studying the role of NR4A2 in this tumor and the effects of NR4A2 ligands such as DIM-C-pPhCl. Results demonstrate that NR4A2 is pro-oncogenic in glioblastoma and the NR4A2 ligands act as antagonists and thus represent a new class of chemotherapeutic agents for treating this deadly disease.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

Cell Lines, Antibodies, and Reagents. 1,1-bis(3′-indolyl)-1-(p-chlorophenyl) methane [DIM-C-pPhCl (C-DIM12)], 1,1-bis(3′-indolyl)-1-(4-chloro-3-trifluoromethylphenyl) methane (3-CF3-4-Cl) and 1,1-bis(3′-indolyl)-1-(4-bromo-2-hydroxyphenyl) methane (2-OH-4-Br) were synthesized in the laboratory. Patient-derived xenografts (PDXs) from human gliomas cell lines 17008, 15037, 14104s, 14015s and 15049 were generated from fresh tumor specimens collected from newly diagnosed patients with no prior chemo- or radiotherapy treatment. Established human malignant glioma cell lines U87-MG, A172, T98G, and CCF-STTG1 were purchased from the American Type Culture Collection (Manassas, VA). PDX cells were maintained in Dulbecco's Modified Eagle's Medium (DMEM)/Hams F-12 50/50 mix supplemented with L-glutamine, 10% fetal bovine serum (FBS), 1× MEM non-essential amino acids, and 10 μg/ml gentamycin (Gibco, Dublin, Ireland). U87-MG, A172, T98G, and CCF-STTG1 were maintained in DMEM1X supplemented with 10% FBS. All cells were maintained at 37° C. in the presence of 5% CO₂, and the solvent (dimethyl sulfoxide; DMSO) used in the experiments was ≤0.2%. DMEM, DMEM F-12 50/50 mix, FBS, formaldehyde, and trypsin were purchased from Sigma-Aldrich (St. Louis, Mo.). Cleaved poly (ADP-ribose) polymerase (cPARP, cat# 9541T), cleaved caspase-8 (cat# 9496T), cleaved caspase-7 (cat# 9491T), Anti-rabbit Alexa Fluor 488 conjugate (cat# 4412s) and Anti-mouse Alexa Fluor 488 conjugate (cat# 4408s) antibodies were obtained from Cell Signaling (Boston, MA); NR4A1 (cat# ab109180) antibody was purchased from Abcam (Cambridge, Mass.); NR4A2 (cat# sc-991), Ki67 (sc-23900) and NR4A3 (cat# sc-133840) antibodies were obtained from Santa Cruz Biotechnology (Santa Cruz, Calif.), and (3-actin (cat# A5316) antibody from Sigma-Aldrich (St. Louis, Mo.). Chemiluminescence reagents (Immobilon Western) for Western blot imaging were purchased from Millipore (Billerica, Mass.). Apoptotic, Necrotic, and Healthy Cells Quantification Kit was purchased from Biotium (Hayward, Calif.), invasion chambers (cat# 354480) was purchased from Corning Inc. (Corning, N.Y.), and XTT cell viability kit was obtained from Cell Signaling (Boston, Mass.). Lipofectamine 2000 was purchased Invitrogen (Carlsbad, Calif.). Luciferase reagent (cat# E1483) was purchased from Promega (Madison, Wis.). Antisense oligonucleotides 3 and 4 that is specific to NR4A2 were purchased from AUM Biotech (Philadelphia, Pa.). The siRNA complexes used in the study that were purchased from Sigma-Aldrich are as follows: siGL2-5′: CGU ACG CGG AAU ACU UCG A (SEQ ID NO: 1), siNR4A1 (SASI_Hs02_00333289), siNR4A2 (SASI_Hs02_00341055) and siNR4A3 (SASI_Hs01_00091655).

Transactivation Assay. Cells (8×10⁴) per well were plated on 12-well plates in DMEM/F-12 supplemented with 2.5% charcoal-stripped FBS and 0.22% sodium bicarbonate. After 24 h growth, various amounts of DNA [i.e., UASₓ₅-Luc (400 ng), GAL4-Nurr1 (40 ng) and β-gal (40 ng)] were cotransfected into each well by Lipofectamine 2000 reagent (Invitrogen, Carlsbad, Calif.) according to the manufacturer's protocol. After 5-6 hr of transfection, cells were treated with plating media (as above) containing either solvent (DMSO) or the indicated concentration of compounds for 24 hr. Cells were then lysed using a freeze-thaw protocol and 30 μL of cell extract was used for luciferase and β-gal assays. LumiCount (Packard, Meriden, Conn.) was used to quantify luciferase and β-gal activities. Luciferase activity values were normalized against corresponding (3-gal activity values as well as protein concentrations determined by Lowry's Method.

Cell Viability Assay. Cells were plated in 96 well plate at a density of 10,000 per well with DMEM F-12 50/50 and DMEM containing 2.5% charcoal-stripped FBS. Cells were treated with DMSO (solvent control) and different concentrations of C-DIM 12, 3-CF₃-4-Cl, and 2-OH-4-Br with DMEM containing 2.5% charcoal-stripped FBS for 0 to 48 hr. After treatment, 25 μL (XTT with 1% of electron coupling solution) was added to each well and incubated for 4 hours as outlined in the manufacturer's instruction (Cell Signaling, Boston, Mass.). Absorbance was measured at wavelength of 450 nm in a 96 well plate reader after incubation for 4 hr in 5% CO₂ at 37 ° C.

Measurement of Apoptosis (Annexin V Staining). Cancer cells were seeded at density of 1.5×10⁵ per ml in 6 well plates and treated with either vehicle (DMSO) or compounds for 24 hr. Cells were then stained and analyzed by flow cytometry using the Dead Cells Apoptosis Kit and Alexa Fluor 488 assay kit according to the manufacturer's protocol (Invitrogen, Carlsbad Calif.).

Scratch and Invasion Assay. 80% confluency was maintained in six-well plates, a scratch was made using a sterile pipette tip and cell migration into the scratch was determined after 24 hr. The BD-Matrigel Invasion Chamber (24-transwell with 8 μm pore size polycarbonate membrane) was used in a modified Boyden chamber assay. The medium in the lower chamber contained the complete culture medium of GBM, which acts as a chemoattractant. PDX cells (5×10⁴ cells/insert) in serum-free medium were plated into the upper chamber with or without various concentrations of compounds and incubated for 24 hr at 37 ° C., 5% CO₂; the non-invading cells were removed from the upper surface of the membrane with a wet cotton swab. 10% formalin was used to fix the invading cells on the lower surface for 10 min, stained in hematoxylin and eosin Y solution (H&E). After washing and drying, the numbers of cells in five adjacent fields of view were counted.

Small Interfering RNA Interference Assay. Cells (2×10⁵ cells/well) were plated in six-well plates in the complete culture medium. After 24 hr, the cells were transfected with 100 nM of each siRNA duplex for 6 hr using Lipofectamine RNAiMAX reagent (Invitrogen, Carlsbad, Calif.) following the manufacturer's protocol. Anti-sense oligonucleotides targeting NR4A2 were used directly in to the 6 well plates and the final concentration was made 10 μM. For siRNA mediated transfection, culture media was changed to the fresh medium containing 10% FBS whereas culture media was not changed for anti-sense oligonucleotides. Both transfection conditions were incubated for 42 hours. After incubation, the cells were treated with either vehicle (DMSO) or different concentrations of the compound and cells were collected for further experiments.

Immunofluorescence. 15037, 14015s and U87-MG cells (1.0×10⁵ per ml) were plated in complete culture media and treated with either DMSO or C-DIM 12 for 24 hr or with siCt or siNR4A2 for 48 hours. Cells were then fixed with 37% formalin, blocked, treated with fluorescent Ki67 primary antibody for 24 hr. Cells were then washed with PBS and treated with anti-mouse IgG Fab2 Alexa Fluor 488 secondary antibody for 2 hr at room temperature. Finally, cells were observed using a Zeiss confocal fluorescence microscope.

Western Blot Analysis. 17008, 15037, 14104s, 14015s, 15049, U87-MG, A172, T98G, and CCF-STTG1 cells were seeded at density of 1.5×10⁵ per ml in 6 well plates and treated with various concentration of compounds and whole cell proteins were extracted using RIPA lysis buffer containing 10 mM Tris-HCl (pH 7.4), 150 mM NaCl, 1 mM ethylenediaminetetraacetic acid (EDTA), 1% Triton X-100 (w/v), 0.5% sodium deoxycholate and 0.1% sodium dodecylsulphate (SDS) with protease and phosphatase inhibitor cocktail. Protein concentrations were measured using Lowry's method and equal amounts of protein were separated in 10% and 15% SDS-PAGE and transferred to a Polyvinylidene difluoride (PVDF) membrane. PVDF membranes were incubated overnight at 4° C. with primary antibodies in 5% skimmed milk and incubated for 2-3 hr with secondary antibodies conjugated with horseradish peroxidase (HRP). Membranes were then exposed to HRP-substrate and immune reacted proteins were detected with chemiluminescence reagent.

Three-Dimensional (3D) Tumor Spheroid Invasion Assay. The cells were suspended in the complete medium (2×10⁴ cells/ml). Spheroids were produced by seeding 200 μl of the cell suspension into a well of a 96-well round-bottomed ultra-low attachment culture plate (Costa, #7007). After incubation at 37° C. in 5% CO₂ incubator for 24 hr, 100 μl/well of growth medium from the spheroid plates was removed and 100 μl/well of Matrigel (Corning, #356234) was added on the bottom of each well. The plate was transferred to the incubator for 1 hr and 100 μl of the complete media containing 3 times the desired final concentration of compounds was supplemented and then incubated for 3-5 days followed by fixation in 4% formaldehyde. Spheroid invasion was determined by measuring the cross-sectional areas of the spheroid center and the rim of invaded cells using ImageJ.

Xenograft Study. Female athymic nu/nu mice (4-6 weeks old) were purchased from Harlan Laboratories (Houston, TX). U87-MG cells (1×10⁶) were harvested in 100 μl of DMEM and suspended in ice-cold Matrigel (1:1 ratio) and s.c. injected to either side of the flank area of the mice. After one week of tumor cell inoculation, mice were divided into two groups of 5 animals each. The first group received 100 μL of vehicle (corn oil), and second group of animals received an injection of 30 mg/kg/day of C-DIM 12 in 100 μl volume of corn oil by i.p. for three weeks. All mice were weighed once a week over the course of treatment to monitor changes in body weight. Tumor volumes could not be determined over the period of treatment because xenografted tumors were relatively deep. After three weeks of treatment, mice were sacrificed and tumor weights were determined.

Statistical Analysis. One way ANOVA and Dunnett's test were used to determine statistical significance between two groups. In order to confirm the reproducibility of the data, the experiments were performed at least three independent times and results were expressed as means ±standard deviation (SD). P-values less than 0.05, were considered to be statistically significant.

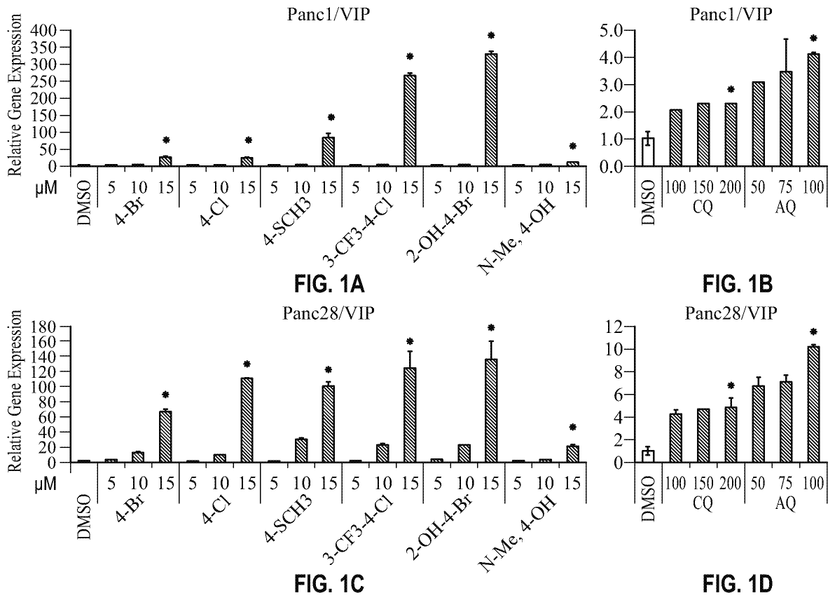

Structure Activity Studies. In structure activity studies two quinoline derivatives chloroquine (CQ) and amodiaquine (AQ) were used as “control” NR4A2 ligands which have previously been studied in models of Parkinson's disease. The effects of bis-indole and quinoline derivatives on activation of the NR4A2 regulated genes vasoactive intestinal peptide (VIP) was investigated. Panc1 (FIG. 1A and FIG. 1B) and Panc28 (FIG. 1C and FIG. 1D) cells were treated with bis-indole analogs (5, 10 and 15 μM), CQ (100, 150 and 200 μM) or AQ (50, 75 and 100 μM). Relative expression levels of VIP were determined by quantitative PCR analysis. Results are expressed as means±SD for at least six separate determinations for each treatment. The asterisk (*) indicates significant gene induction (P<0.01) of the highest concentration treatment versus solvent control (DMSO). FIG. 1A shows that the bis-indole compounds significantly induce VIP in Panc1 cells with up to a 330-fold induction observed using 2-OH-4-Br (FIG. 1A). Although CQ and AQ induced VIP in Panc1 cells (2.3 to 4.1-fold), the magnitude of the response was significantly lower than the NR4A2-active bis-indole compounds (FIG. 1B). The differences between bis-indoles and quinolines for induction of VIP in Panc28 cells (FIG. 1C and FIG. 1D, respectively) were similar to those observed in Panc1 cells. The least active bis-indole compound, N—Me-4-OH, was a more potent inducer of VIP in Panc1 and Panc28 cells than either CQ or AQ. Thus, new NR4A2 ligands more potent than DIM-C-pPhCl (4-Cl) and DIM-C-pPhBr (4-Br) have been identified.

Quenching of NR4A1 Tryptophan Fluorescence by Direct Ligand Binding. Tryptophan fluorescence spectra were obtained as described herein. Briefly, histidine-tagged ligand binding domain (LBD) of NR4A1 at a final concentration of 0.5 μmol/L in 1.0 mL of phosphate-buffered saline (PBS, pH 7.4) was used for fluorescence measurements. The protein was incubated for 3 min at 25 oC in a temperature-controlled (Quantum Northwest TC125) fluorescence spectrophotometer (Varian Cary Eclipse). The fluorescence spectrum was obtained using an excitation wavelength of 285 nm (excitation slit width=5 nm) and an emission wavelength range of 300-420 nm (emission slit width=5 nm). Aliquots (0.1 μL/aliquot) of ligand (10 mmol ligand/L ethanol) were then added to the cuvette containing NR4A1 to a final ligand concentration of 30 μmol ligand/L. After each aliquot of ligand, the NR4A1/ligand solution was incubated at 25° C. for 3 min and the NR4A1 tryptophan fluorescence was measure as described above. Addition of ethanol only (up to final volume of 3.0 μL) had no effect on NR4A1 tryptophan fluorescence (data not shown). Ligand binding affinity (Kd) to NR4A1 was determined by measuring NR4A1 tryptophan fluorescence intensity at emission wavelength of 330 nm according to the references listed above. Ligand only fluorescence intensity at each ligand concentration was used to correct the NR4A1 tryptophan fluorescence intensity as described in the above references.

Ligand Binding to NR4A1 LBD: bisANS Displacement Assay. bisANS (Molecular Probes, Inc/ThermoFisher) is essentially non-fluorescent in aqueous solution; however, bisANS fluorescence increases significantly upon binding to protein such as NR4A1. The binding affinity (Kd) and binding stoichiometry (Bmax) of NR4A1/bisANS were determined essentially as described in FEBS Journal, 2014, 281, 2266-2283. For NR4A1/bisANS the binding affinity (Kd) was determined to be 0.84 μmon and the binding stoichiometry (Bmax) was determined to be 0.80 mol bisANS/mol NR4A1. The ability of ligand to displace bisANS was analyzed essentially as described in FEBS Journal, 2014, 281, 2266-2283. Briefly, histidine-tagged NR4A1 LBD (final concentration=0.05 μmon PBS, pH 7.4) was incubated at 25° C. for 3 min in the presence of 5.0 μmol bisANS/L. The bisANS fluorescence spectrum was obtained utilizing a Varian Cary Eclipse fluorescence spectrophotometer with an excitation wavelength of 365 nm (excitation slit width=5 nm) and an emission wavelength range of 400-600 nm (emission slit width=5 nm). Ligand titration was accomplished as described above for direct ligand binding using a stock ligand concentration of 10 mmol ligand/L ethanol. Addition of ethanol only had no effect on NR4A1/bisANS fluorescence (data not shown). Ligand binding affinity (Ki) to NR4A1 was determined by measuring NR4A1/bisANS fluorescence intensity at emission wavelength of 500 nm according to the above reference. Ligand/bisANS fluorescence intensity at each ligand concentration was used to correct the NR4A1/bisANS/ligand fluorescence intensity as described in the above reference.

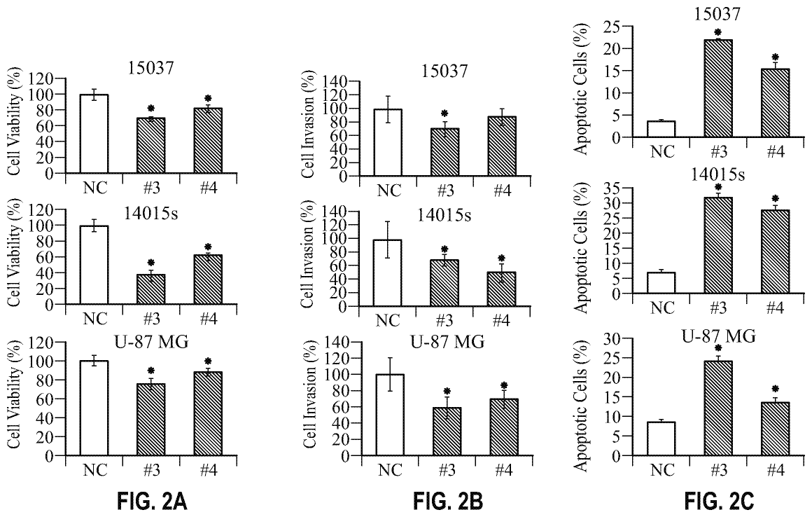

The expression of NR4A receptors in glioblastoma cell lines (A172, U87-MG, U98G and CCF-STTG1) and patient-derived cells (1708, 15037, 14004s, 14015s and 15049) was determined by western blot analysis of whole cell lysates. NR4A2 was expressed in all cell lines and NR4A3 was expressed in most of the cell lines, whereas NR4A1 was detected in the established cell lines, but only in two of the patient-derived cell lines. Thus, the patient-derived cell lines are somewhat unique in the expression of NR4A2 in the absence of NR4A1. In the present disclosure, the role of NR4A2 in U87-MG, 15037 and 14015s cells were investigated to determine the effects of NR4A2 knockdown using antisense oligonucleotides (#3 and #4) on cell proliferation, survival and invasion. NR4A2 knockdown was conducted as follows: Glioblastoma cells were transfected with oligonucleotide targeting NR4A2 (siNR4A2-#3 and #4) or a non-specific control (NC) and whole cell lysates were analyzed by western blots and effects on cell proliferation (FIG. 2A) cell invasion (FIG. 2B) and Annexin V staining (FIG. 2C) were determined as outlined herein. Cells were transfected with a non-specific control (NC) or oligonucleotides (#3 and #4) targeting NR4A2 and markers of apoptosis were determined by western blots of whole cell lysates. Results (FIGS. 2A-2C) are means±SD for at least three determinations per treatment groups and significant (P<0.05) effects [compared to control (NC)] are indicated (*). Caspase 8 cleavage was not observed in 15037 cells. Antisense oligonucleotides were effective in decreasing expression of NR4A2 in 15037, 14015s and U87-MG cells and this was accompanied by decreased cell proliferation (FIG. 2A) and invasion using a Boyden chamber assay (FIG. 2B). Moreover, decreased expression of NR4A2 induced markers of apoptosis including induction of Annexin V staining (FIG. 2C) and cleavage of caspase 8, 7 and PARP in 15037, 14015s and U87-MG cells (note: cleaved caspase 8 was not detected in 15037 cells).

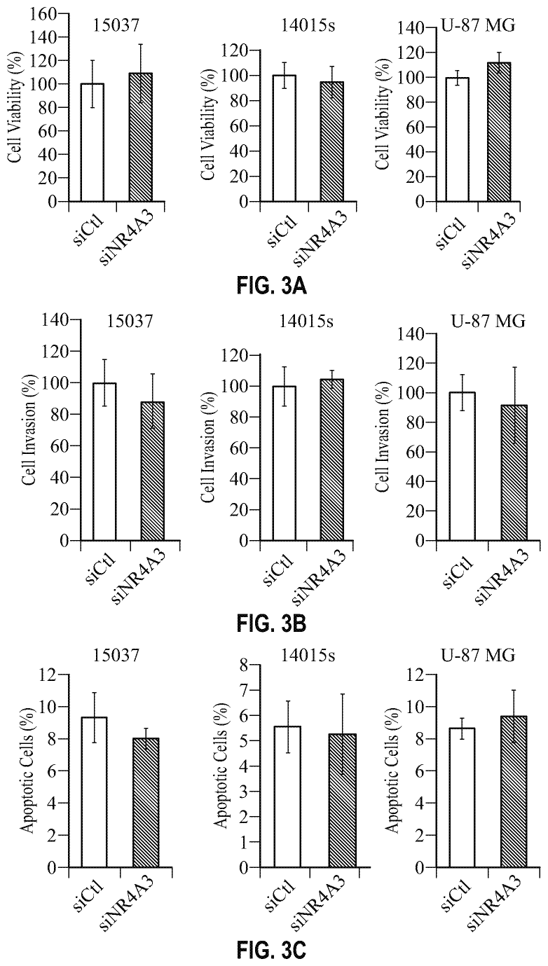

Since 15037, 14015s and U87-MG cells express NR4A3, the effects of NR4A3 knockdown by RNA interference (RNAi) on the phenotypic characteristics of the cell lines was also investigated. Cells were transfected with NC or oligonucleotides targeting NR4A3 and effects on NR4A3 expression (determined by western blots of whole cell lysates), cell proliferation (FIG. 3A), cell invasion (FIG. 3B) and Annexin V staining (FIG. 3C) as outlined herein. Cells were transfected with siNR4A2 or siNR4A3 and Ki67 staining was determining as outlined herein. Results (FIGS. 3A-3C) are expressed as means±SD at least three determinations per-treatment group and significant (P<0.05) differences with control/untreated groups are indicated (*). Loss of NR4A3 had minimal effects on cell proliferation (FIG. 3A), invasion (FIG. 3B), or apoptosis (FIG. 3C), and staining for the Ki67 proliferation marker demonstrated that the loss of NR4A3 had minimal effects on Ki67 staining. These results clearly demonstrate for the first time that NR4A2 is a pro-oncogenic factor in glioblastoma cells, whereas NR4A3 has minimal effects on their growth, survival and invasion.

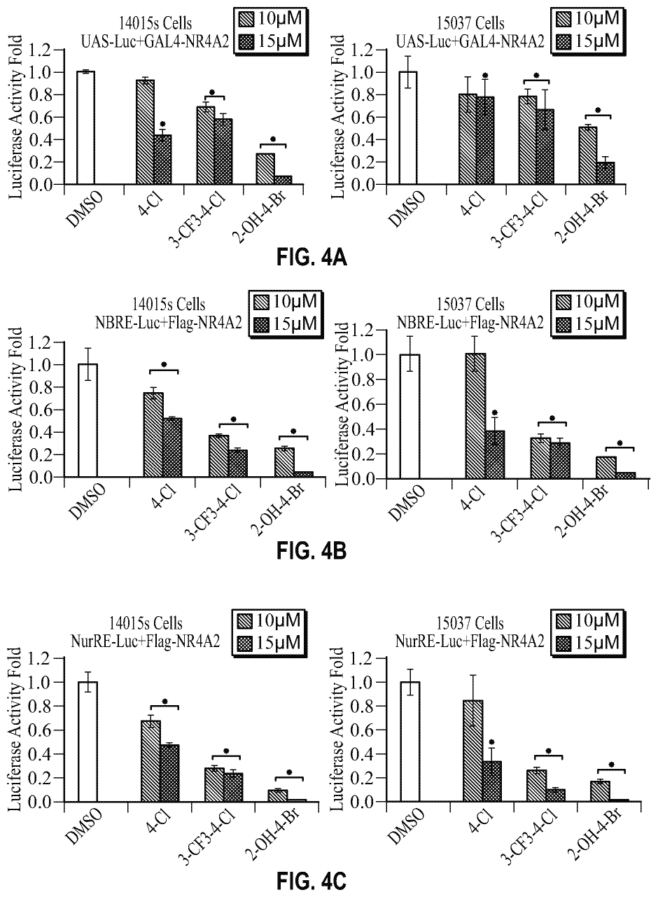

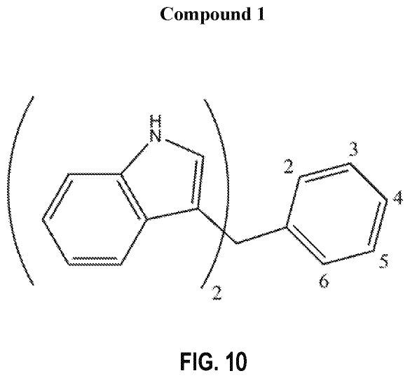

Previous studies have identified a series of bis-indole-derived compounds (C-DIMs) that induce NR4A2-dependent transactivation, and 1,1-bis(3′-indolyl)-1-(p-chlorophenyl)methane (DIM-C-pPhCl, 4-Cl) has been used as a prototypical NR4A2 ligand (Compound 1). Compound 1, illustrated below and depicted in FIG. 10, shows a prototypical NR4A2 ligand. Cells were treated with NR4A2 ligands. Cells were transfected with UAS-Luc/GAL4-NR4A2 (FIG. 4A), NBRE-Luc/NR4A2 expression plasmid (40 ng) (FIG. 4B), and NurRE-Luc/NR4A2 expression plasmid (40 ng) (FIG. 4C), treated with bis-indole-derived ligand and luciferase activity was determined as outlined herein. Results are means±SD for three replicate determinations for each treatment group and significant (P<0.05) effects (compared to DMSO control) are indicated (*).

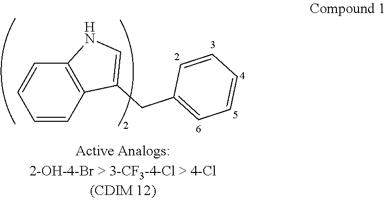

Ongoing screening in pancreatic cancer cells identified 3 additional NR4A2 ligands, including 1,1-bis(3′-indolyl)-1-(4-chloro-3-trifluoromethylphenyl)methane (3-CF₃-4-Cl), 1,1-dimethyl-1,1-bis(3′-indolyl)-1-(p-hydroxyphenyl)methane (N—Me-4-OH), and 1,1-bis(3′-indolyl)-1-(4-bromo-2-hydroxy-phenyl)methane (2-OH-4-Br). These compounds induced NR4A2-dependent transactivation in Panc1 cells; however, in 14015s and 15037 glioblastoma cells transfected with a GAL4-NR4A2 chimera and a reporter plasmid containing GAL4 response elements (UAS5-luc), all of these compounds decreased transactivation (luciferase activity) (FIG. 4A). Ligand-dependent modulation of NR4A2-regulated gene expression was also investigated using reported gene constructs containing an NGF1-B response element-luciferase construct (NBRE₃-luc) (FIG. 4B) and a Nur-responsive element (NuRE₃-luc) (FIG. 4C) which bind NR4A2 as a monomer and dimer, respectively. The three ligands also decreased NR4A2-dependent transactivation in these assays, suggesting that they act as NR4A2 inverse agonist/antagonist in glioblastoma cells.

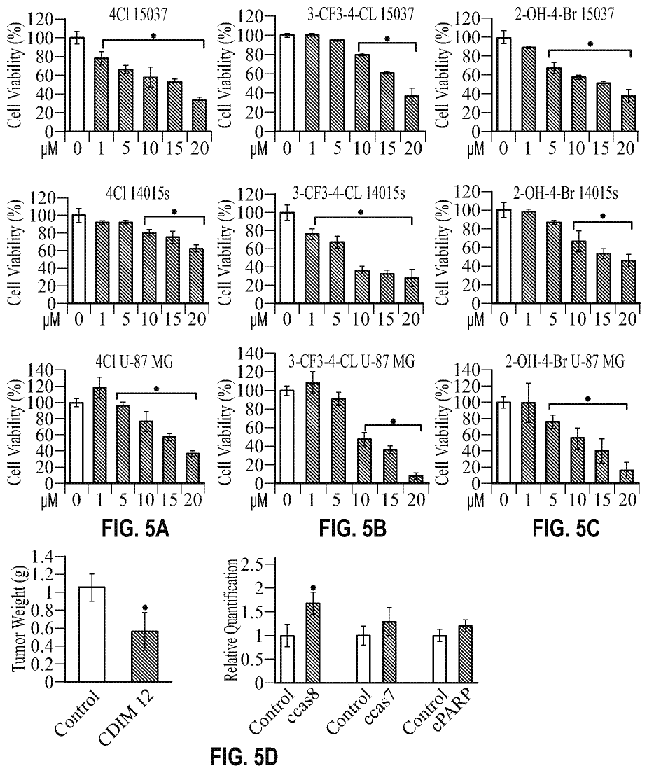

Treatment of 15037, 14015s, and U87-MG cells with DIM-C-pPhCl (FIG. 5A), 3-CF₃-4-Cl (FIG. 5B), and 2-OH-4-Br (FIG. 5C) inhibited cell proliferation. Cells were treated with different concentration of DIM-C-pPhCl (4-Cl) (FIG. 5A), 1,1-bis(3′-indolyl)-1-(4-chloro-3 trifluoromethylphenyl) methane (3-CF₃-4-Cl) (FIG. 5B), and 1,1-bis(3′-indolyl)-1-(4-bromo-2-hydroxyphenyl) methane (2-OH-4-Br) (FIG. 5C) and effects on glioblastoma cell proliferation were determined as outlined herein. Athymic nude mice bearing U87-MG cells as xenografts were treated with 4-Cl (30 mg/kg/day) and effects on tumor weights and expression of apoptosis markers in tumor from control (corn oil) and 4-Cl-treated mice were determined by western blot analysis of tumor lysates. Expression levels of various proteins in control versus 4-Cl-treated mice were determined (normalized to β-actin). Glioblastoma cells were treated with different NR4A2 antagonists and Ki-67 staining was determined as outlined herein. Results (FIGS. 5A-5C) are expressed as means±SD for at least three replicates for each treatment group and significant (P<0.05) difference from untreated controls are indicated (*).

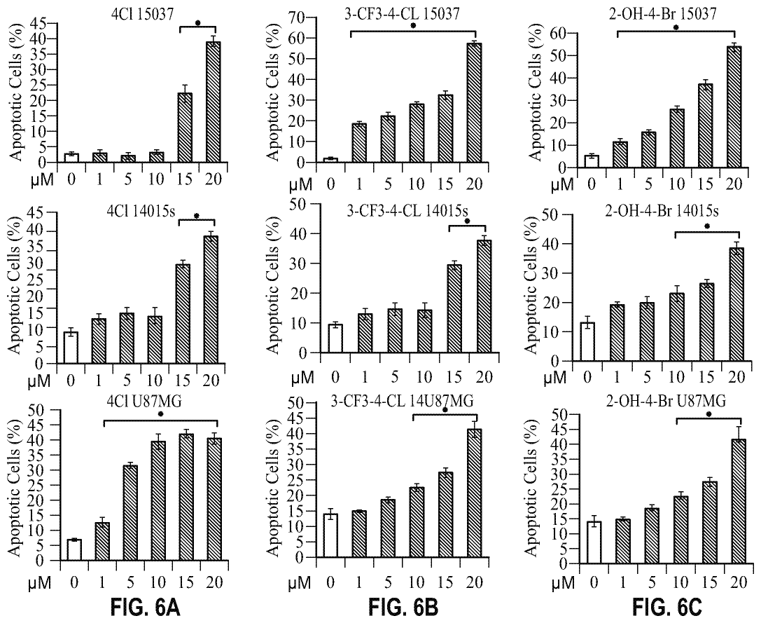

It was observed that 4-Cl (30 mg/kg/day) significantly decreased tumor weight in athymic nude mice bearing U87-MG tumor cells as a xenograft (FIG. 5D), and this was accompanied by significant upregulation of cleaved caspase 8 but not cleaved caspase 7 and cPARP in tumors from 4-Cl treated mice compared with vehicle controls. Treatment with 4-Cl for 24 hours decreased Ki67 (proliferation marker) in 15037, 14015s and U87-MG cells. Thus, the NR4A2 ligands and NR4A2 knockdown (FIGS. 2A-2C) were growth inhibitory, indicating that the C-DIMs are NR4A2 antagonists and this is consistent with their antagonist activities in the transactivation assays (FIGS. 4A-4C). Treatment of glioblastoma cells with 4-Cl (FIG. 6A), 3-CF₃-4-Cl (FIG. 6B) and 2-OH-4-Br (FIG. 6C) induced Annexin V staining and cleaved caspase 7, 8 and PARP cleavage. Glioblastoma cells were treated with different concentrations of 4-Cl (FIG. 6A), 3-CF₃-4-Cl (FIG. 6B), and 2-OH-4-Br (FIG. 6C) and effects on induction of Annexin V were determined as outlined herein. Glioblastoma cells were treated with different concentrations of NR4A2 antagonists and whole cell lysates were analyzed for markers of apoptosis by western blots. Results (FIGS. 6A-6C) were expressed as means±SD for at least three determinations per treatment group and significant (P<0.05) responses compared to untreated controls are indicated (*). These results were comparable to those observed after knockdown of NR4A2 (FIG. 2C).

The potency of the various ligands in terms of growth inhibition and induction of apoptosis was ligand-, cell type-, and response-dependent with the most obvious difference in the fold induction of Annexin V in 15037 (high) versus 14015s (low) cells, and this was due, in part, to the relatively higher expression of Annexin V in untreated 14015s cells.

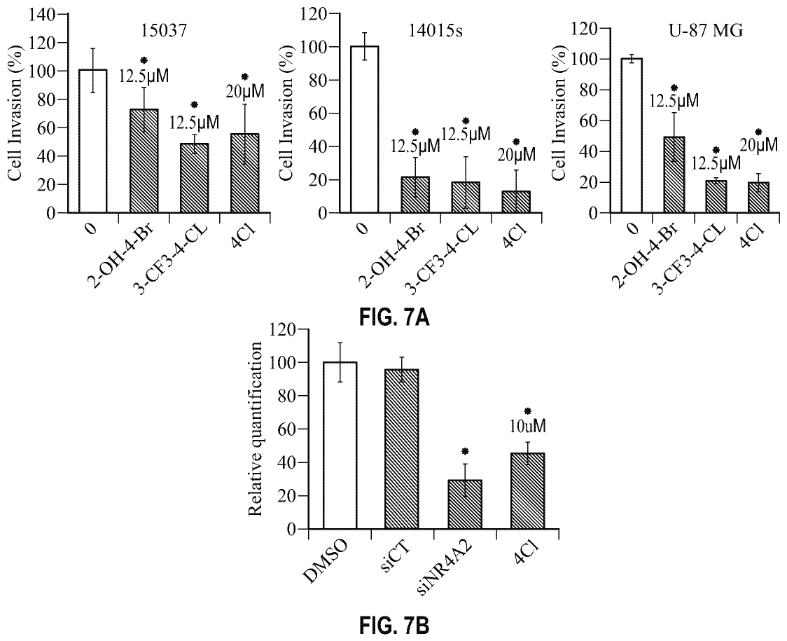

Cells were treated with 12.5 μM (3-CF₃-4-Cl and 2-OH-4-Br) or 20 μM (C-DIM 12) and effects of glioblastoma cell invasion or migration in Boyden chamber and scratch assay respectively as outlined herein. NR4A2 ligands as inhibition of cell migration in a tumor spheroid invasion assay in 15037 cells were also determined as outlined herein (note: 14015s and U87-MG cells did not exhibit invasion in this assay). The NR4A1 antagonists also inhibited invasion of 15037 and 14015s cells in a Boyden chamber assay where the latter cell line appeared to be more sensitive, and 4-Cl-mediated inhibition of cell invasion required higher concentrations compared to 3-CF₃-4-Cl or 2-OH-4-Br (FIG. 7A). Similar results were observed in scratch assays in 15037 and 14015s cells where 20 μM DIM-C-pPhCl exhibited minimal inhibition of migration and lower concentrations (12.5 μM) of 2-OH-4-Br and 3-CF₃-4-Cl inhibited migration with the latter compound being the most potent inhibitor. It was also observed that knockdown of NR4A2 or treatment with 10 μ,M 4-Cl inhibited tumor spheroid invasion using 15037 cells compared to DMSO (solvent control) or cells transfected with a control oligonucleotide (siCt) (FIG. 7B). These results demonstrate that NR4A2 is a growth promoting, survival and pro-invasion gene in glioblastoma, and C-DIM/NR4A2 ligands act as NR4A2 antagonists and represent a novel chemotherapeutic approach for treatment of this disease.

It is estimated that 23,880 new cases of cancer of the brain and nervous system will be diagnosed, and 16,380 deaths will occur from these diseases. GBM is the most frequently diagnosed malignant brain tumor, and global incidence of this disease varies from 0.59-3.69 per 100,000. A diagnosis of GBM in an adult is devastating since patient survival times are in the range of 12-15 months and the 3-year survival of patients after diagnosis is in the 3-5% range. Primary de novo GBMs constitute approximately 90% of all cases and occur in elderly patients, whereas secondary GBMs are mainly diagnosed in younger patients. GBM is a complex disease that involves multiple genetic alterations including mutations of several genes, resulting in a highly aggressive disease that is difficult to treat. The current standard-of-care for newly diagnosed glioblastoma patients, include surgery, adjuvant radiotherapy and the drug temozolomide (TMZ; an alkylating agent), and these treatment regimens have had limited success. The most troubling biological characteristics of high-grade glioma cells are their propensity and capacity to invade into the normal surrounding brain tissue, thereby evading the surgeon's knife as well as the radiation delivered to the surgical resection margin. This reservoir of infiltrating tumor cells form a subpopulation of glioma stem cells that become a major source of tumor recurrence/progression, and they are typically resistant to chemoradiation, and are frequently the cause of eventual patient mortality. The orphan nuclear receptor NR4A2 plays an important role in neuronal function, and previous studies show that 4-Cl and some related C-DIM compounds cross the blood-brain barrier and inhibit NR4A2-dependent inflammatory responses in mouse models of Parkinson's disease. Results of preliminary studies in established and patient-derived glioblastoma cell lines demonstrate expression of NR4A1, NR4A2, and NR4A3 in these cells and the patient-derived cells primarily expressed NR4A2/NR4A3 with relatively low levels of NR4A1. The differential expression of these orphan receptors in patient-derived cells afforded the opportunity to investigate the function of NR4A2 and the potential for targeting this receptor as a novel approach for treating GBM patients.

A gene knockdown approach was initially used for determining the functions of NR4A2 in patient-derived 14015s, 15037 and U87-MG glioblastoma cells. The results indicated that loss of NR4A2 resulted in inhibition of growth, induction of apoptosis, and inhibition of invasion. The effects of NR4A2 knockdown were in contrast to results obtained after knockdown of NR4A3, which had minimal effects on cell growth, survival, and migration. Thus, NR4A2 clearly exhibits pro-oncogenic activity in GBM and these results were consistent with previous reports on the function of NR4A2 in other cancer cell lines and the pro-oncogenic activity of NR4A2.

Previous studies have characterized 4-Cl as an NR4A2 ligand that is effective as an anti-inflammatory drug in treating some NR4A2-regulated pathways in models of Parkinson's disease. In transactivation studies in pancreatic cancer cells, 4-Cl activated NR4A2-dependent transactivation, whereas 4-Cl and two additional C-DIM analogs inhibited NR4A2-dependent transactivation in GBM cells (FIGS. 4A-4C). Thus, in terms of NR4A2-dependent transactivation, 4-Cl and related compounds are selective receptor modulators that exhibit cell type-specific agonist and antagonist activities, and this has previously been observed for C-DIMs that bind NR4A1.

4-Cl and related compounds not only inhibit NR4A2-dependent transactivation, but also NR4A2-dependent cell growth, survival, and migration. Moreover, similar responses were observed in athymic nude mice using U87-MG cells in a xenograft model where 4-Cl inhibited tumor growth and induced apoptosis in the tumors.

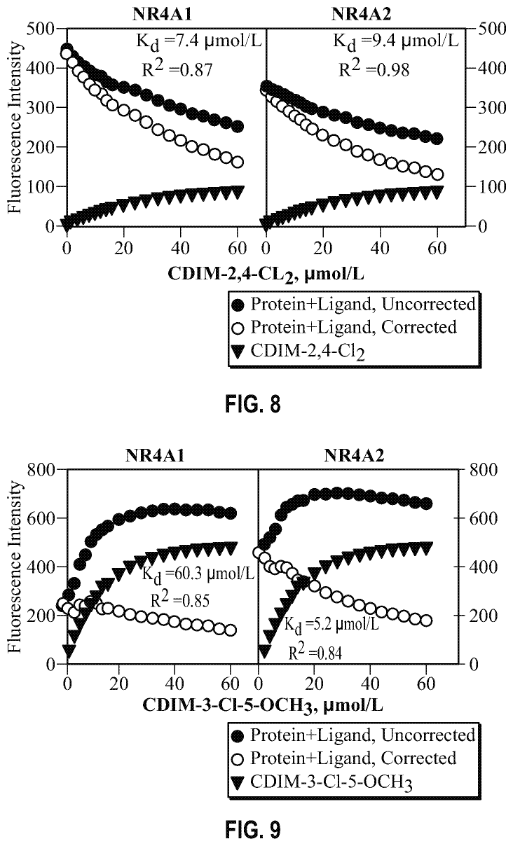

These results confirm the pro-oncogenic activity of NR4A2 and show that NR4A2 ligands such as the C-DIMs that act as antagonists represent a novel approach for treating GBM. Studies focused on investigating and identifying NR4A2-regulated genes/pathways in GBM and also developing more potent NR4A2 antagonists for future clinical applications are readily envisioned. Additionally, and as detailed in the preceding, C-DIM12 (4-chloro analog) targets NR4A2 through interactions with the coactivator binding site and not the ligand binding domain of NR4A2. Accordingly, ligand binding assay for NR4A1 and NR4A2 which measures ligand-induced quenching of tryptophan fluorescence by the ligand were developed to further characterize activity with respect to NR4A1 and NR4A2. A tryptophane residue is located in the ligand binding domain (LBD) of both receptors and the LBD of the receptor is used in the binding assay. Table 1, shown below, summarizes the binding K_{D} values of C-DIM12 and other NR4A2-active compounds and they do not quench the LBD-Trp and this confirms previous modeling studies showing that C-DIM12 binds NR4A2 outside the LBD of NR4A2. The 3-Cl and 2-Cl-phenyl isomers of C-DIM12 gave similar results (Table 1). However, introduction of a second chlorine substituent into the phenyl ring results in binding (tryptophan quenching) of several dichloro-substituted analog to both NR4A2 and NR4A1 (FIG. 8). These unexpected results suggested the possibility that CDIMs containing two or more substituents on the phenyl ring bind both NR4A2 and NR4A1. This hypothesis was tested using two series of compounds; 3,5-disubtituted analogs of the prototypical NR4A1 ligand, C-DIM8 [1,1-bis(3′-indolyl)-1-(p-hydroxyphenyemethane] (Table 2, shown below); and a new set of 3,5-disubstituted phenyl CDIM analogs (Table 3, shown below) (FIG. 9). The results show that all of these compounds with two or more phenyl substituents bind NR4A2 and NR4A1.

TABLE 1
Binding of 4-substituted (phenyl ring) and dichloro-
substituted CDIMs to NR4A1 and NR4A2.

Tryptophan Quenching
K_{d}, mmol/L

| Ligand | NR4A1 | NR4A2 |
|---|---|---|
| CDIM-4-Cl | Not Calculated | Not Calculated |
| CDIM-4-Br | Not Calculated | Not Calculated |
| CDIM-4-OCF₃ | Not Calculated | Not Calculated |
| CDIM-4-SCH₃ | Not Calculated | Not Calculated |
| CDIM-2-Cl | Not Calculated | Not Calculated |
| CDIM-3-Cl | Not Calculated | Not Calculated |
| CDIM-2,4-Cl₂ | 7.4 | 9.4 |
| CDIM-2,5-Cl₂ | 11.1 | 9.2 |
| CDIM-2,6-Cl₂ | 8.2 | 14.5 |
| CDIM-3,4-Cl₂ | 12.2 | 16.4 |

TABLE 2
Binding of 3,5-disubstituted analogs of CDIM8 (1,1-bis(3′-indolyl)-
1-(p-hydroxyphenyl)methane) to NR4A1 and NR4A2

Tryptophan Quenching
K_{d}, mmol/L

| Ligand | NR4A1 | NR4A2 |
|---|---|---|
| CDIM8 | 0.56 | 2.0 |
| CDIM8-3,5-(CH₃)₂ | 24.5 | 10.7 |
| CDIM8-3,5-Br₂ | 1.3 | 7.4 |
| CDIM8-3,5-Cl₂ | 4.1 | 13.9 |
| CDIM8-3-Br-5-OCH₃ | 6.7 | 7.3 |
| CDIM8-3-Cl-5-OCH₃ | 6.6 | 2.2 |
| CDIM8-3-Cl-5-Br | 4.2 | 11.1 |
| CDIM8-3-Cl-5-F | 3.6 | 1.1 |

TABLE 3
Binding of 3,5-disubstituted analogs
of CDIM to NR4A1 and NR4A2

Tryptophan Quenching
K_{d}, mmol/L

| Ligand | NR4A1 | NR4A2 |
|---|---|---|
| CDIM-3,5-Br₂ | 6.5 | 12.2 |
| CDIM-2,5-Br₂ | 8.5 | 7.8 |
| CDIM-3,5-Cl₂ | 7.7 | 12.0 |
| CDIM-3,5-(CH₃)₂ | 133 | 79 |
| CDIM-3-Br-5-OCF₃ | 4.8 | 7.9 |
| CDIM-3-Br-5-OCH₃ | 1.8 | 3.5 |
| CDIM-3-Cl-5-OCH₃ | 60.3 | 5.2 |
| CDIM-3-Cl-5-OCF₃ | 2.3 | 3.5 |
| CDIM-3-Cl-5-CF₃ | 3.1 | 5.5 |

These data highlight promising results for CDIMs with two or more substituents on the phenyl ring as ligands for NR4A2 and NR4A1. Accordingly, the bis-indole-derived compounds, for example, a bis-indole-derived compound with two or more substituents on the phenyl ring can be utilized for inducing anticancer or anti-inflammatory activity in cells, as well as treating other diseases, such as, but not limited to, cancer, brain cancer, breast cancer, kidney cancer, colon cancer, pancreatic cancer, lung cancer, an inflammatory disease, asthma, chronic peptic ulcers, tuberculosis, rheumatoid arthritis, periodontitis, ulcerative colitis, Crohn's disease, sinusitis, active hepatitis, and combinations thereof, as the bis-indole-derived compounds are capable of binding to NR4A1 and NR4A2.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group.

The terms “a”, “an”, and other singular terms are intended to include the plural forms thereof unless specifically excluded.

1. A method of treating a disease by induction of activity in cells, the method comprising administering a bis-indole-derived compound to a subject in need thereof.

2. The method of claim 1, further comprising binding, by the bis-indole-derived compound, to at least one of nuclear receptor 4A1 (NR4A1) and nuclear receptor 4A2 (NR4A2).

3. The method of claim 1, wherein the induction of activity in the cells is at least one of anticancer activity and anti-inflammatory activity.

4. The method of claim 1, wherein the bis-indole-derived compound (CDIM) comprises two or more substituents on a phenyl ring thereof.

5. The method of claim 1, wherein the bis-indole-derived compound is selected from the group consisting of 1,1-bis(3′-indolyl)-1-(p-chlorophenyl)methane (DIM-C-pPhCl; 4-Cl), 1,1-bis(3′-indolyl)-1-(4-chloro-3-trifluoromethylphenyl)methane (3-CF₃-4-Cl), 1,1-dimethyl-1,1-bis(3′-indolyl)-1-(p-hydroxyphenyl)methane (N—Me-4-OH), 1,1-bis(3′-indolyl)-1-(4-bromo-2-hydroxy-phenyl)methane (2-OH-4-Br), 1-bis(3′indolyl)-1-(p-bromophenyl)methane (DIM-C-pPhBr), 1,1-bis(3′-indolyl)-1-(p-hydroxyphenyl)methane (CDIM8), a 3,5-disubstituted analog of CDIM8, CDIM8-3,5-(CH₃)₂, CDIM8-3,5-Br₂, CDIM8-3,5-Cl₂, CDIM8-3-Br-5-OCH₃, CDIM8-3-Cl-5-OCH₃, CDIM8-3-Cl-5-Br, CDIM8-3-Cl-5-F, CDIM, a 3,5-disubstituted analog of CDIM, CDIM-3,5-Br₂, CDIM-2,5-Br₂, CDIM-3,5-Cl₂, CDIM-3,5-(CH₃)₂, CDIM-3-Br-5-OCF₃, CDIM-3-Br-5-OCH₃, CDIM-3-Cl-5-OCH₃, CDIM-3-Cl-5-OCF₃, CDIM-3-Cl-5-CF₃, and combinations thereof.

6. The method of claim 1, wherein the bis-indole-derived compound performs a function on the cells selected from the group consisting of inducing NR4A1-dependent transactivation in the cells, inducing NR4A2-dependent transactivation in the cells, inhibiting growth of the cells, inducing apoptosis in the cells, inhibiting survival of the cells, inhibiting migration of the cells, and combinations thereof.

7. The method of claim 1, wherein the cells are selected from the group consisting of A172, U87-MG, U98G, CCF-STTG1, 1708, 15037, 14004s, 14015s, 15049, glioblastoma multiforme (GBM) cells, and combinations thereof.

8. The method of claim 1, wherein the bis-indole-derived compound is at least one of a bis-indole-derived NR4A1 ligand and a bis-indole-derived NR4A2 ligand.

9. The method of claim 8, wherein the at least one of the bis-indole-derived NR4A1 ligand and the bis-indole-derived NR4A2 ligand performs a function selected from the group consisting of antagonizing NR4A1 in the cells, targeting NR4A1 in the cells, antagonizing NR4A2 in the cells, targeting NR4A2 in the cells, and combinations thereof.

10. The method of claim 1, wherein the cells comprise at least one of NR4A1 and NR4A2 in cancer cells.

11. The method of claim 10, wherein the cancer cells correspond to a cancer selected from the group consisting of brain cancer, breast cancer, kidney cancer, colon cancer, pancreatic cancer, lung cancer, and combinations thereof.

12. The method of claim 1, wherein the disease is selected from the group consisting of cancer, brain cancer, breast cancer, kidney cancer, colon cancer, pancreatic cancer, lung cancer, an inflammatory disease, asthma, chronic peptic ulcers, tuberculosis, rheumatoid arthritis, periodontitis, ulcerative colitis, Crohn's disease, sinusitis, active hepatitis, and combinations thereof.

13-32. (canceled)

33. A compound for treating a disease by induction of activity in cells, the compound comprising a bis-indole-derived compound.

34. The compound of claim 33, wherein the bis-indole-derived compound binds to at least one of nuclear receptor 4A1 (NR4A1) and nuclear receptor 4A2 (NR4A2).

35. The compound of claim 32, wherein the induction of activity in the cells is at least one of anticancer activity and anti-inflammatory activity.

36. The compound of claim 33, wherein the bis-indole-derived compound (CDIM) comprises two or more substituents on a phenyl ring thereof.

37. The compound of claim 33, wherein the bis-indole-derived compound is selected from the group consisting of 1,1-bis(3′-indolyl)-1-(p-chlorophenyl)methane (DIM-C-pPhCl; 4-Cl), 1,1-bis(3′-indolyl)-1-(4-chloro-3-trifluoromethylphenyl)methane (3-CF₃-4-Cl), 1,1-dimethyl-1,1-bis(3′-indolyl)-1-(p-hydroxyphenyl)methane (N—Me-4-OH), 1,1-bis(3′-indolyl)-1-(4-bromo-2-hydroxy-phenyl)methane (2-OH-4-Br), 1-bis(3′indolyl)-1-(p-bromophenyl)methane (DIM-C-pPhBr), 1,1-bis(3′-indolyl)-1-(p-hydroxyphenyl)methane (CDIM8), a 3,5-disubstituted analog of CDIM8, CDIM8-3,5-(CH₃)₂, CDIM8-3,5-Br₂, CDIM8-3,5-Cl₂, CDIM8-3-Br-5-OCH₃, CDIM8-3-Cl-5-OCH₃, CDIM8-3-Cl-5-Br, CDIM8-3-Cl-5-F, CDIM, a 3,5-disubstituted analog of CDIM, CDIM-3,5-Br₂, CDIM-2,5-Br₂, CDIM-3,5-Cl₂, CDIM-3,5-(CH₃)₂, CDIM-3-Br-5-OCF₃, CDIM-3-Br-5-OCH₃, CDIM-3-Cl-5-OCH₃, CDIM-3-Cl-5-OCF₃, CDIM-3-Cl-5-CF₃, and combinations thereof.

38. The compound of claim 33, wherein the bis-indole-derived compound performs a function selected from the group consisting of inducing NR4A1-dependent transactivation in cells, NR4A2-dependent transactivation in cells, inhibiting growth of cells, inducing apoptosis in cells, inhibiting survival of cells, inhibiting migration of cells, and combinations thereof.

39. The compound of claim 38, wherein the cells are selected from the group consisting of A172, U87-MG, U98G, CCF-STTG1, 1708, 15037, 14004s, 14015s, 15049, glioblastoma multiforme (GBM) cells, and combinations thereof.

40. The compound of claim 38, wherein the cells comprise at least one of NRA1 and NR4A2 in cells.

41. The compound of claim 38, wherein the cells are cancer cells.

42. The compound of claim 41, wherein the cancer cells are selected from the group consisting of brain cancer cells, breast cancer cells, kidney cancer cells, colon cancer cells, pancreatic cancer cells, lung cancer cells, and combinations thereof.

43. The compound of claim 33, wherein the bis-indole-derived compound is at least one of a bis-indole-derived NR4A1 ligand and a bis-indole-derived NR4A2 ligand.

44. The compound of claim 43, wherein the at least one of the bis-indole-derived NR4A1 ligand and the bis-indole-derived NR4A2 ligand performs a function selected from the group consisting of antagonizing NR4A1, targeting NR4A1, antagonizing NR4A2, targeting NR4A2, and combinations thereof.

45. The compound of claim 33, wherein the disease is selected from the group consisting of cancer, brain cancer, breast cancer, kidney cancer, colon cancer, pancreatic cancer, lung cancer, an inflammatory disease, asthma, chronic peptic ulcers, tuberculosis, rheumatoid arthritis, periodontitis, ulcerative colitis, Crohn's disease, sinusitis, active hepatitis, and combinations thereof.
